(19) United States
(12) Patent Application Publication
(10) Pub. No.: US 2013/0289650 A1
(43) Pub. Date: Oct. 31, 2013

(54) Neuromodulation for Hypertension Control

(71) Applicants: Andreas Karlsson, Solna (SE); Stuart Rosenberg, Castaic, CA (US); John Gustafsson, Hagersten (SE)

(72) Inventors: Andreas Karlsson, Solna (SE); Stuart Rosenberg, Castaic, CA (US); John Gustafsson, Hagersten (SE)

(73) Assignee: PACESETTER, INC., Sylmar, CA (US)

(21) Appl. No.: 13/456,140

(22) Filed: Apr. 25, 2012

(51) Int. Cl. A61N 1/36

(52) U.S. Cl. 607/44

(57) ABSTRACT

Neuromodulation for controlling hypertension and other cardio-renal disorders of a patient is disclosed. A neuromodulation device is configured to be delivered to a patient's body and to apply an electric activation to decrease renal sympathetic hyperactivity of the patient based on monitored blood pressure of the patient, substantially without thermal energization of the patient's body by applying the electric activation. The electric activation may also depend on monitored blood volume of the patient. A feedback control module may be used to provide feedback control information for adjusting the electric activation based on the monitored blood pressure and volume of the patient.

### BACKGROUND

The present invention relates generally to neuromodulation, and more specifically to neuromodulation for treating hypertension and other cardio-renal disorders of a patient.

Hypertension (HTN), or high blood pressure (HBP), is defined as a consistently elevated blood pressure (BP) greater than or equal to 140 mmHg systolic blood pressure (SBP) and 90 mmHg diastolic blood pressure (DBP). Hypertension is a “silent killer” that is not associated with any symptoms and in 95% of cases (primary hypertension) the specific cause is unknown. In the remaining 5% of patients (secondary hypertension), specific causes including chronic kidney disease, diseases of the adrenal gland, coarctation of the aorta, thyroid dysfunction, alcohol addiction, pregnancy or the use of birth control pills are present. In secondary hypertension, when the root cause is treated, blood pressure usually returns to normal.

Hypertension is a disease that affects 74.5 million patients in the United States alone. Worldwide, 26.4% of the adult population had hypertension in the year 2000 and 29.2% is predicted to have this condition by the year 2025. This corresponds to 972 million in the year 2000: 333 million in economically developed countries and 639 million in economically developing countries. The number of adults with hypertension in 2025 is predicted to increase by about 60% to a total of 1.56 billion. Geographically, most of this rise can be attributed to an expected increase in the number of people with hypertension in economically developing regions. Although the number of people with hypertension in economically developed countries is predicted to increase by 24% from 333 million to 413 million, a rise of 80% is predicted for economically developing countries from 639 million to 1.15 billion.

Out of the world's adult hypertension population, 5-10% suffers from truly resistant hypertension. Resistant hypertension is defined as failure to achieve goal blood pressure when a patient adheres to the maximum tolerated doses of 3 antihypertensive drugs including a diuretic. Hyperactivity of the sympathetic nervous system serving the kidneys is associated with hypertension and its progression, as well as with chronic kidney disease and heart failure. The resistant hypertension population, although only a portion of the growing hypertensive population, is still huge. Needless to say, there is a need for additional therapeutic options for this class of unsuccessfully treated patients. The resistant hypertension population is a target population for this innovation.

It is generally accepted that the causes of hypertension are multi-factorial, with a significant factor being the chronic hyper-activation of the sympathetic nervous system (SNS), especially the renal sympathetic nerves. Renal sympathetic efferent and afferent nerves, which lie in the wall of the renal artery, have been recognized as a critical factor in the initiation and maintenance of systemic hypertension. Renal arteries, like all major blood vessels, are innervated by perivascular sympathetic nerves that traverse the length of the arteries. The perivascular nerves consist of a network of axons, terminals, and varicosities, which are distributed mostly in the medial-adventitial and adventitial layers of the arterial wall.

Signals coming in to the kidney travel along efferent nerve pathways and influence renal blood flow, trigger fluid retention, and activate the renin-angiotensin-aldosterone system cascade. Renin is a precursor to the production of angiotensin II, which is a potent vasoconstrictor, while aldosterone regulates how the kidneys process and retain sodium. All of these mechanisms serve to increase blood pressure. Signals coming out of the kidney travel along afferent nerve pathways integrated within the central nervous system, and lead to increased systemic sympathetic nerve activation. Chronic over-activation can result in vascular and myocardial hypertrophy and insulin resistance, causing heart failure and kidney disease.

Previous clinical studies have documented that denervating the kidney has a positive effect for both hypertension and heart failure patients. However, given the highly invasive and traumatic nature of the procedure and the advent of more effective antihypertensive agents, the procedure was not widely employed.

More recently, catheter ablation has been used for renal sympathetic denervation. Renal denervation is a method whereby amplified sympathetic activities are suppressed to treat hypertension or other cardiovascular disorders and chronic renal diseases. The objective of renal denervation is to neutralize the effect of renal sympathetic system which is involved in arterial hypertension. The renal sympathetic efferent and afferent nerves lie within and immediately adjacent to the wall of the renal artery. Energy is delivered via a catheter to ablate the renal nerves in the right and left renal arteries in order to disrupt the chronic activation process. As expected, early results appear both to confirm the important role of renal sympathetic nerves in resistant hypertension and to suggest that renal sympathetic denervation could be of therapeutic benefit in this patient population.

In clinical studies, therapeutic renal sympathetic denervation has produced predictable, significant, and sustained reductions in blood pressure in patients with resistant hypertension. Catheters are flexible, tubular devices that are widely used by physicians performing medical procedures to gain access into interior regions of the body. A catheter device can be used for ablating renal sympathetic nerves in therapeutic renal sympathetic denervation to achieve reductions of blood pressure in patients suffering from renal sympathetic hyperactivity associated with hypertension and its progression. Renal artery ablation for afferent and efferent denervation has been shown to substantially reduce hypertension.

Spinal cord stimulation (SCS) is a widely used clinical technique to treat ischemic pain in peripheral, cardiac and cerebral vascular diseases. The use of this treatment advanced rapidly during the late 80's and 90's, particularly in Europe. Although the clinical benefits of SCS are clear and the success rate remains high, the mechanisms are not yet completely understood. Experimental studies in animal models suggest that SCS at lumbar spinal segments (L2-L3) produces vasodilation in the lower limbs and feet which is mediated by antidromic activation of sensory fibers and release of vasoactive substances, and decreased sympathetic outflow. Also at C3-C6, SCS induces increased blood flow, this time in the upper extremities.

### BRIEF SUMMARY

Embodiments of the invention provide neuromodulation for controlling hypertension and other cardio-renal disorders of a patient. In specific embodiments, by modulating the lower thoracic and upper lumbar spinal cord (where the renal sympathetic innervation originates) or by stimulating the neurons closer to the kidneys such as the renal plexus or even closer to the kidneys such as the neurons along the renal artery, it is proposed that sympathetic inhibition and vasodilation, which have been shown when treating peripheral disease, would here affect the kidneys. The neuromodulation of the kidneys, at any level as described (spinal cord, renal plexus, or direct renal nerve) will control blood pressure in hypertensive patients. This blood pressure control is through three mechanisms: (1) sympathetic inhibition of the renal innervation, (2) antidromic activation at the renal/kidney level and release of vasoactive substances which in turn increases renal blood flow, and (3) antidromic activation at the periphery to reduce the systemic vascular resistance. These mechanisms would contribute to decreased renal sympathetic hyperactivity, affecting the downstream messaging including RAAS (renin-angiotensin-aldosterone system) and thereby reducing systemic hypertension. Furthermore, an increase in renal blood flow would increase the renal clearance (total volume of solute cleared), even at constant filtration rate (i.e., constant gradient in concentration/osmolarity). This would also be a contributing factor to lowering elevated blood pressure levels, in this case by modulating blood volumes. This treatment could then be used to treat the drug resistant hypertension population.

This invention does not use thermal energy but employs electric energy for neuromodulation without thermal energization of the patient's body. This distinguishes the invention over U.S. Pat. No. 7,717,948 which discloses thermally induced renal neuromodulation via direct and/or indirect application of thermal energy. The thermal delivery in the '948 patent may be ablative or nonablative, but relies on electroporation resulting from thermally induced neuromodulation. The patient's condition relating to hypertension or other cardio-renal disorders (e.g., blood pressure, blood volume, etc.) is monitored and used as feedback to control delivery of the neural modulation via the neural modulator. The monitoring device may be implanted in the patient or may be an external device.

In accordance with an aspect of the present invention, a method of treating hypertension comprises: monitoring a blood pressure of a patient; delivering a neuromodulation device to a patient's body; and applying an electric activation using the neuromodulation device to decrease renal sympathetic hyperactivity of the patient based on the monitored blood pressure of the patient, substantially without thermal energization of the patient's body by applying the electric activation.

In some embodiments, the method further comprises: selecting at least one target of the following three targets to which the electric activation is applied to activate: (i) a region of a spinal cord responsible for renal innervation, or (ii) neurons in a vicinity of a kidney, or (iii) a region of the spinal cord or a peripheral nerve to produce vasodilation with reduced systemic vascular resistance; and based on the selected at least one target, determining at least one of a plurality of electric activation parameters to be used in applying the electric activation, including a current level, a pulse width, a frequency, a duty cycle, and a location of the patient's body to which the electric activation is applied.

In specific embodiments, the method further comprises: selecting at least one of the following three types of electric activation to be applied: (i) sympathetic inhibition of renal innervation, or (ii) antidromic activation at a kidney level of the patient to increase renal blood flow, or (iii) antidromic activation at a periphery of the patient to reduce systemic vascular resistance; and based on the selected type of electric activation, determining at least one of a plurality of electric activation parameters to be used in applying the electric activation, including a current level, a pulse width, a frequency, a duty cycle, and a location of the patient's body to which the electric activation is applied.

In some embodiments, the method further comprises adjusting the electric activation based on the monitored blood pressure of the patient. Adjusting the electric activation comprises modifying at least one of a plurality of electric activation parameters including a current level, a pulse width, a frequency, a duty cycle, and a location of the patient's body to which the electric activation is applied. The method further comprises monitoring a blood volume of the patient; and adjusting the electric activation based on the monitored blood pressure and the monitored blood volume of the patient. Adjusting the electric activation may comprise modifying at least one of a plurality of electric activation parameters including a current level, a pulse width, a frequency, a duty cycle, and a location of the patient's body to which the electric activation is applied. Adjusting the electric activation may comprise referring to a lookup table which provides electric activation plans for different conditions of monitored blood pressure and monitored blood volume, the electric activation plans each specifying a setting of at least one of a plurality of electric activation parameters including a current level, a pulse width, a frequency, a duty cycle, and a location of the patient's body to which the electric activation is applied. The electric activation is applied until the monitored blood pressure falls within a preset blood pressure range and the monitored blood volume falls within a preset blood volume range for the patient.

In accordance with another aspect of the invention, a system of treating hypertension comprises a neuromodulation device configured to be delivered to a patient's body and to apply an electric activation to decrease renal sympathetic hyperactivity of the patient based on monitored blood pressure of the patient, substantially without thermal energization of the patient's body by applying the electric activation.

In some embodiments, the system further comprises a processor; a memory; and a feedback control module to provide feedback control information for adjusting the electric activation to be applied by the neuromodulation device based on the monitored blood pressure of the patient. Adjusting the electric activation comprises modifying at least one of a plurality of electric activation parameters including a current level, a pulse width, a frequency, a duty cycle, and a location of the patient's body to which the electric activation is applied.

In specific embodiments, the neuromodulation device is configured to apply the electric activation to decrease renal sympathetic hyperactivity of the patient based on monitored blood pressure and monitored blood volume of the patient, substantially without thermal energization of the patient's body by applying the electric activation. The feedback control module provides feedback control information for adjusting the electric activation to be applied by the neuromodulation device based on the monitored blood pressure and monitored blood volume of the patient. The neuromodulation device is configured to apply the electric activation until the monitored blood pressure falls within a preset blood pressure range and the monitored blood volume falls within a preset blood volume range for the patient.

In accordance with another aspect of this invention, a computer-readable storage medium storing a plurality of instructions for controlling a data processor to provide treatment for hypertension of a patient via a neuromodulation device. The plurality of instructions comprise instructions that cause the data processor to apply an electric activation using the neuromodulation device to decrease renal sympathetic hyperactivity of the patient based on monitored blood pressure of the patient, substantially without thermal energization of the patient's body by applying the electric activation.

These and other features and advantages of the present invention will become apparent to those of ordinary skill in the art in view of the following detailed description of the specific embodiments.

### BRIEF DESCRIPTION OF THE DRAWINGS

FIG. 1 is an illustration of the innervation of the kidneys.

FIG. 2 is an illustration of sympathetic pathways between the sympathetic control centers and the kidneys.

FIG. 3 is a schematic diagram illustrating an example of a neuromodulation device.

FIG. 4 depicts an exemplary embodiment of a neurostimulator implanted in the torso of a patient.

FIG. 5 is a high level block diagram of a neuromodulation device of FIG. 3 according to an embodiment for creating complex and/or multi-purpose stimulation sets.

FIG. 6 is a schematic block diagram depicting an exemplary embodiment of a controller for use in the neuromodulation device of FIG. 5.

FIG. 7 is a schematic block diagram depicting an exemplary embodiment of a system as seen in FIG. 5.

FIG. 8 is a high level block diagram illustrating another example of a neuromodulation system.

FIG. 9 is a schematic diagram illustrating an example of a feedback system for controlling the neural modulation to treat hypertension based on results of a monitoring device.

FIG. 10 shows an example of a neuromodulation delivery table that summarizes the various scenarios expected to be encountered and the system response for delivery of neuromodulation.

FIG. 11 is a flow diagram illustrating an example of feedback control of neuromodulation for hypertension control.

### DETAILED DESCRIPTION

In the following detailed description of the invention, reference is made to the accompanying drawings which form a part of the disclosure, and in which are shown by way of illustration, and not of limitation, exemplary embodiments by which the invention may be practiced. In the drawings, like numerals describe substantially similar components throughout the several views. Further, it should be noted that while the detailed description provides various exemplary embodiments, as described below and as illustrated in the drawings, the present invention is not limited to the embodiments described and illustrated herein, but can extend to other embodiments, as would be known or as would become known to those skilled in the art. Reference in the specification to “one embodiment”, “this embodiment”, or “these embodiments” means that a particular feature, structure, or characteristic described in connection with the embodiment is included in at least one embodiment of the invention, and the appearances of these phrases in various places in the specification are not necessarily all referring to the same embodiment. Additionally, in the following detailed description, numerous specific details are set forth in order to provide a thorough understanding of the present invention. However, it will be apparent to one of ordinary skill in the art that these specific details may not all be needed to practice the present invention. In other circumstances, well-known structures, materials, circuits, processes and interfaces have not been described in detail, and/or may be illustrated in block diagram form, so as to not unnecessarily obscure the present invention.

In the following description, relative orientation and placement terminology, such as the terms horizontal, vertical, left, right, top and bottom, is used. It will be appreciated that these terms refer to relative directions and placement in a two dimensional layout with respect to a given orientation of the layout. For a different orientation of the layout, different relative orientation and placement terms may be used to describe the same objects or operations.

Furthermore, some portions of the detailed description that follow are presented in terms of algorithms, flow-charts and symbolic representations of operations within a computer. These algorithmic descriptions and symbolic representations are the means used by those skilled in the data processing arts to most effectively convey the essence of their innovations to others skilled in the art. An algorithm is a series of defined steps leading to a desired end state or result which can be represented by a flow chart. In the present invention, the steps carried out require physical manipulations of tangible quantities for achieving a tangible result. Usually, though not necessarily, these quantities take the form of electrical or magnetic signals or instructions capable of being stored, transferred, combined, compared, and otherwise manipulated. It has proven convenient at times, principally for reasons of common usage, to refer to these signals as bits, values, elements, symbols, characters, terms, numbers, instructions, or the like. It should be borne in mind, however, that all of these and similar terms are to be associated with the appropriate physical quantities and are merely convenient labels applied to these quantities. Unless specifically stated otherwise, as apparent from the following discussion, it is appreciated that throughout the description, discussions utilizing terms such as “processing,” “computing,” “calculating,” “determining,” “displaying,” or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

The present invention also relates to an apparatus for performing the operations herein. This apparatus may be specially constructed for the required purposes, or it may include one or more general-purpose computers selectively activated or reconfigured by one or more computer programs. Such computer programs may be stored in a computer-readable storage medium, such as, but not limited to optical disks, magnetic disks, read-only memories, random access memories, solid state devices and drives, or any other types of media suitable for storing electronic information. The algorithms and displays presented herein are not inherently related to any particular computer or other apparatus. Various general-purpose systems may be used with programs and modules in accordance with the teachings herein, or it may prove convenient to construct a more specialized apparatus to perform desired method steps. In addition, the present invention is not described with reference to any particular programming language. It will be appreciated that a variety of programming languages may be used to implement the teachings of the invention as described herein. The instructions of the programming language(s) may be executed by one or more processing devices, e.g., central processing units (CPUs), processors, or controllers.

Exemplary embodiments of the invention, as will be described in greater detail below, provide apparatuses and methods for neuromodulation for hypertension control. More specifically, hypertension is treated by controlling the blood pressure by introducing a neuromodulation device into the body of a patient and applying an electric field to cause the overall lowered sympathetic drive or decreased renal sympathetic hyperactivity in the patient, substantially without thermal energization of the patient's body by the application of the electric field. While the thermal energization is ideally zero due to the low level of electrical power delivered to the patient's body, it is understood herein that the thermal energization is so negligible that it is nonexistent for practical purposes but a rise in temperature of at most about 0.5° C. in the region of applying the electric field would be acceptable and fall within the scope of substantially without thermal energization. The electrical power is a function of the amount of current and waveform parameters such as pulse width, frequency, and duty cycle.

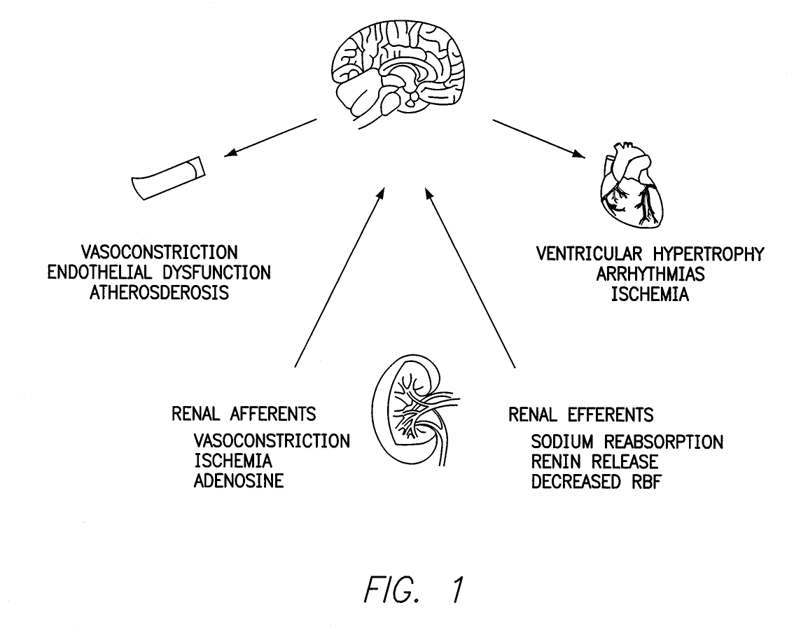

FIG. 1 is an illustration of the innervation of the kidneys. The sympathetic innervation of the kidneys originates from the lower thoracic and upper lumbar spinal cord regions and the parasympathetic innervation of the kidneys originates from the vagus nerve. Several lines of evidence indicate the presence of a sympathetic hyperactivity in chronic renal failure, and its relationship with arterial hypertension. It is suggested that diseased kidneys send afferent nervous signals to central integrative sympathetic nuclei, thus contributing to the development and maintenance of arterial hypertension. The nerve/neural modulation for controlling blood pressure works via one of three mechanisms of action as described herein below.

Neural Modulation

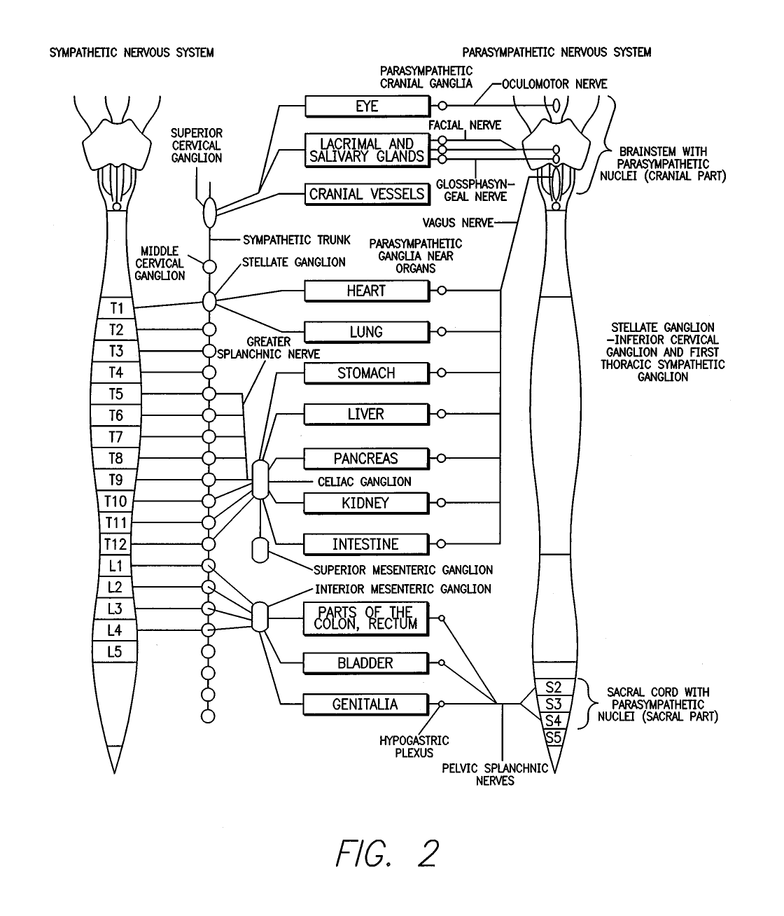

FIG. 2 is an illustration of sympathetic pathways between the sympathetic control centers and the kidneys. Afferent sympathetic pathways travel from the kidney to the control centers for neuromodulation in the midbrain. Activation of these pathways increases global sympathetic traffic, which may adversely affect vascular tone and integrity, and may lead to inappropriate myocardial hypertrophy, myocardial cell damage, and arrhythmias. Increased renal sympathetic signaling stimulates sodium retention, volume expansion, and renal vasoconstriction. The consequences of increased renal sympathetic efferent traffic may also lead to an increase in afferent traffic, thereby creating a positive feedback loop with many deleterious vascular, myocardial, and renal consequences.

A first mechanism of action of this invention is to reduce the afferent sympathetic reflex from the kidneys by blocking sympathetic input to the kidneys. Spinal cord modulation, renal plexus modulation, or renal nerve modulation are directed toward blocking such efferent sympathetic drive to the kidneys to prevent further signal transduction that regulates systemic hypertensive response. This can be done by orthodromic neural stimulation of a subset of nerves or by sympathetic inhibition, both modulating local reflex at the kidneys to prevent sympathetic afferent signaling.

Orthodromic neural stimulation at an intermediate frequency (e.g., about 50-500 Hz) involves titrating current level and waveform parameters to achieve the desired effects. The pulse width is generally in the intermediate range (about 100 μs to 2 ms) and the current level depends on the location of the electrode(s) with respect to the nerve. For instance, if the electrode is in intimate contact with the renal nerve or plexus, the current level will be relatively low; if the electrode location is in the epidural space of the spinal cord (i.e., indirect nerve contact), the current level will be relatively high. These parameters will be titrated.

Sympathetic inhibition to block the efferent sympathetic drive to the kidneys can be achieved using different ranges of electric field parameters. One example is direct current (DC) stimulation. Because long term DC stimulation may lead to permanent nerve damage, it is desirable to limit the duty cycle to avoid permanent nerve damage (e.g., about 50-70%, or alternately turning on a DC step pulse for several seconds and then turning it off for the same or slightly shorter duration). Another example is very high frequency stimulation (e.g., on the order of about 2,000-100,000 Hz), which may necessitate a bias to avoid negative current. The electric fields block orthodromic signaling on the nerves, thereby inhibiting their actions.

A second mechanism of action of this invention is to vasodilate the renal vasculature at the renal/kidney level. Renal artery stenosis is the narrowing of the renal artery, most often caused by atherosclerosis or fibromuscular dysplasia. This narrowing of the renal artery can impede blood flow to the target kidney. Hypertension and atrophy of the affected kidney may result from renal artery stenosis, ultimately leading to renal failure if not treated.

Under the second mechanism, the vasodilation according to one embodiment occurs in response to antidromic activation brought about by electrical nerve stimulation at the renal/kidney level. While vasodilation may not have profound effects in stenosis of atherosclerotic origin, it will help open vessels that are contracting in response to global signaling from RAAS and other pathways. Renal/kidney vasodilation (i.e., directly of the renal nerve) occurs by modulation of the RAAS. Alternatively, the vasodilation according to another embodiment occurs in response to antidromic activation of the nerves (i.e., activation in the reverse direction) that stimulates post-synaptic neurons to release neurotransmitters from the post-synaptic terminals, thereby producing the effect of vasodilation at the level of the kidney, primarily of the smaller vascular beds in the renal cortex. It also has hemodynamic renal clearance effect. In either case, the antidromic activation triggers local processing at the level of the kidney. It is hypothesized that the increased renal blood flow would increase the renal clearance, also at a constant filtration rate (i.e., at constant differential solute concentration on either side of the nephrons of the kidney), while the local processing alters the neural signaling of the kidney back to the central and distributed processing centers of the nervous system, primarily via modulation of the RAAS. Thus, this method of decreasing blood pressure has two synergistic, contributing methods: first, modulation of renal neural signaling particularly decreased renal sympathetic activity, and second, renal vasodilation leading to decreased solute concentration and ultimately restoration of systemic euvolemia.

Antidromic activation at the renal/kidney level can be produced at an intermediate frequency (e.g., about 50-500 Hz) and an intermediate pulse width (e.g., about 100 μs to 2 ms). The current level depends on the location of the electrode(s) with respect to the nerve. For instance, if the electrode is in intimate contact with the renal nerve or plexus, the current level will be relatively low (e.g., about 0.1-2 milli-amp); if the electrode location is in the epidural space of the spinal cord (i.e., indirect nerve contact), the current level will be relatively high (e.g., about 1-20 milli-amp). In general, antidromic activation of a particular nerve requires higher current level than orthodromic activation of the same nerve, given other electrical parameters are unchanged. Those skilled in the art will appreciate that methods exist for sequentially delivering electrical stimuli from a plurality of electrodes arrayed along the length of a nerve in order to preferentially activate a nerve in the antidromic direction at a given current level.

A third mechanism of action of this invention is to vasodilate at the vascular beds of the lower body (i.e., guts and legs). This would also be a contributing factor to lowering elevated blood pressure levels, by directly altering the resistance against which generated blood pressure must pass. Antidromic activation is applied to a peripheral nerve (e.g., mesenteric nerve or sciatica nerve) or in the lower lumbar region of the spinal cord. It produces hemodynamic effect in reducing the systemic vascular resistance. In this third mechanism of peripheral vasodilation, the end result of decreased blood pressure is brought about by direct hemodynamic changes within systemic vasculature.

Specific embodiments of the present invention employ these three mechanisms for hypertension control: (1) decreased sympathetic outflow, (2) antidromic activation at the renal/kidney level, and (3) antidromic activation at the periphery. Under the first and second mechanisms, by modulating the lower thoracic and upper lumbar spinal cord (responsible for the renal innervation) or by stimulating the neurons closer to the kidneys such as the renal plexus or even closer to the kidneys such as the neurons along the renal artery, it is here proposed that the same mechanisms as were at work when treating peripheral disease would affect the kidney, namely, modulation of integration of neural signaling, and specifically in this case renal signaling and the RAAS pathway. Also under the second mechanism, hemodynamic changes at the level of the kidney serve to increase renal blood flow and thus renal clearance, ultimately leading to restoration of euvolemia. Under the third mechanism, by stimulating the lower lumbar spinal cord or the peripheral nerves, a decrease in resistance in large vascular beds of the lower body directly lower arterial blood pressure. Both sympathetic inhibition of the renal innervation and increasing the renal blood flow by antidromic activation would contribute to decrease renal sympathetic hyperactivity. The decreased renal sympathetic hyperactivity, in turn, would result in decreased global hypertensive state due to overall lowered sympathetic drive, with the end result of lowered blood pressure.

Neuromodulation Device

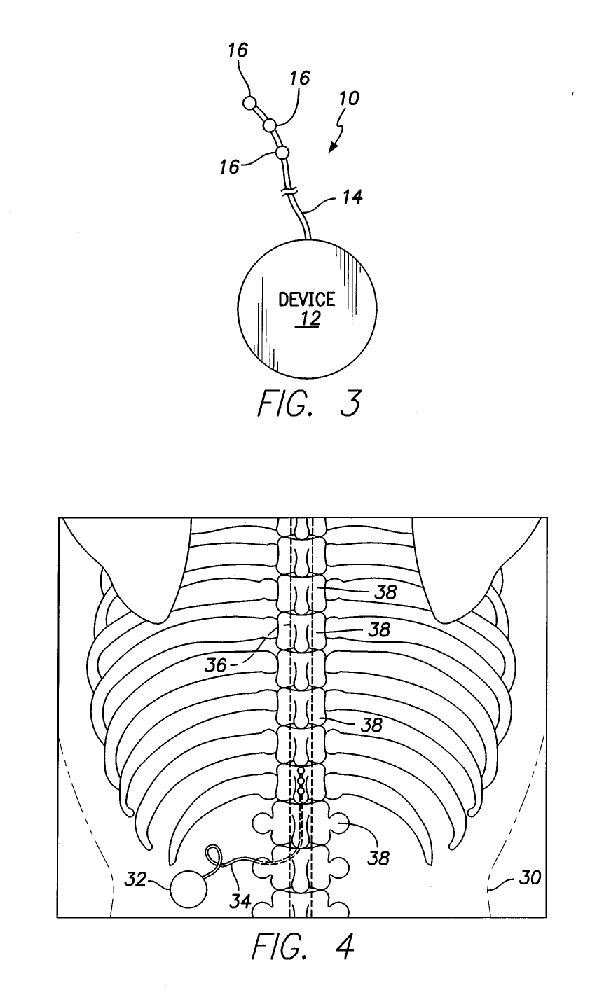

FIG. 3 is a schematic diagram illustrating an example of a neuromodulation system. FIG. 3 shows an implantable neuromodulation system 10 including a modulation or stimulation device 12 that may be implanted in a patient. Attached to the device 12 is a lead 14, which terminates in a set or array of electrodes 16. The device 12 may take various forms, including implanted pulse generators and neurostimulators, among others. The lead 14 and electrodes 16 may take various forms, including cylindrical leads and electrodes, paddles, and lamitrodes, among others. The lead 14 may have one or more electrodes 16 and these electrodes 16 may be shaped in accordance with various functions. Furthermore, more than one lead 14 may be attached to the device 12.

The stimulation device 12 may be configured to stimulate one or more sets of electrodes with one or more pulses having various pulse characteristics. Together, the sets of electrodes and pulse characteristics make stimulation settings. For each stimulation setting, each electrode is set as an anode (+), cathode (−), or neutral (off). These electrode settings are combined with pulse characteristics and pulse patterns to produce stimulation. In specific embodiments, the device 12 is used to stimulate spinal nervous tissue.

FIG. 4 depicts an exemplary embodiment of a neurostimulator implanted in the torso 30 of a patient. In this exemplary embodiment, the stimulation device 32 is installed such that the lead 34 extends into the spinal foramen 36 as defined by the vertebrae 38. The lead 34 terminates with one or more electrodes. These electrodes are used to stimulate or modulate nervous tissue. The positions of the electrodes depend on the desired modulation. As discussed above, the first and second mechanism of action may be achieved by modulating the lower thoracic and upper lumber spinal cord (responsible for the renal innervation), while the third mechanism of action may be achieved by modulating the lower lumber spinal cord. The modulation will depend on both the location and stimulation characteristics of the electric field pulses delivered by device 32. Note that FIG. 4 shows only one example of neuromodulation along the spinal cord. As described above, other ways of modulation are possible including, for instance, modulation in the vicinity of renal plexus or renal artery nerves.

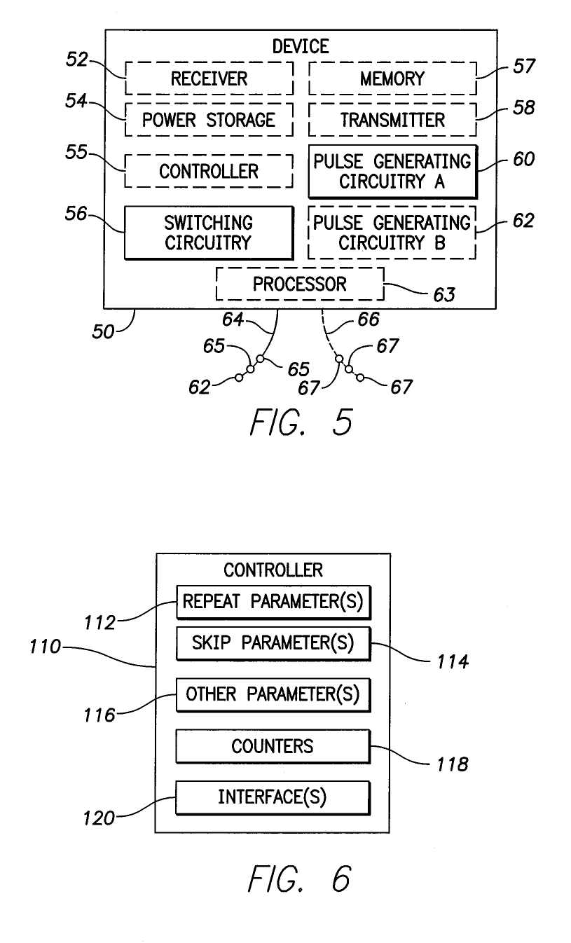

FIG. 5 is a high level block diagram of a neuromodulation device of FIG. 3 according to an embodiment for creating complex and/or multi-purpose stimulation sets. The device 50 has a receiver 52, a transmitter 58, a power storage 54, a controller 55, a switching circuitry 56, a memory 57, pulse generators 60 and 62, and a processor 63. The device 50 is typically coupled to one or more leads 64 and 66. The leads 64 and 66 terminate in one or more electrodes 65 and 67, respectively. The receiver 52 may include a circuitry, an antenna, a coil, or the like. The transmitter 58 may include a circuitry, an antenna, a coil, or the like. The power storage 54 may include various batteries, such as primary cell (i.e., non-rechargeable) batteries or rechargeable batteries (e.g., to be recharged with a charging module via RF). The controller 55 may include any suitable mechanisms or units for modulating and controlling pulses and signals, and may be implemented as software, hardware, or a combination of software and hardware. The switching circuitry 56 may include various contacts, relays, switch matrices, or the like. Further, the switching circuitry 56 in combination with the microprocessor 63 and/or controller 55 may function to drop, skip, or repeat stimulation patterns. The memory 57 may include various forms of random access memory, read-only memory, flash memory, or the like. The memory 57 may be accessible by the controller 55, the switching circuitry 56, and/or the processor 63. The memory 57 may store various stimulation settings, repetition parameters, skipping parameters, programs, instruction sets, and other parameters. The processor 63 may include logic circuitry or microprocessors, or the like. The processor 63 may function to monitor, deliver, and control delivery of the modulation or stimulation signal. Further, the processor 63 may manipulate the switching circuitry 56. This manipulation may or may not be in conjunction with the controller 55. The one or more pulse generators 60 and 62 may include a clock driven circuitry, an oscillating circuitry, or the like. The pulse generator(s) 60 and 62 may deliver an electric or electromagnetic signal through the switching circuitry 56 to the leads 64 and 66 and electrodes 65 and 67. The signal may be modulated by circuitry associated with the switching circuitry 56, controller 55, and/or processor 63 to manipulate characteristics of the signal including amplitude, frequency, polarity, pulse width, of the like.

In one exemplary embodiment, the microprocessor 63 may interact with the switching circuitry 56 to establish electrode configurations. The pulse generator 60, 62 may then generate a pulse and, in combination with the microprocessor 63 and switching circuitry 56, stimulate the tissue with a pulse having the desired characteristics. The controller 55 may interact with the microprocessor 63 and switching circuitry 56 to direct the repetition of the pulse. Alternately, the switching circuitry 56 may be reconfigured to subsequent stimulation settings in an array of stimulation settings. The controller 55 may then direct the skipping or with settings in the array of settings for one or more passes through the stimulation setting array. The controller 55 may be implemented as software for use by the microprocessor 63 or in hardware for interaction with the microprocessor 63 and switching circuitry 56.

FIG. 6 is a schematic block diagram depicting an exemplary embodiment of a controller for use in the neuromodulation device of FIG. 5. The controller 110 may have one or more repeat parameters 112, one or more skip parameters 114, other parameters 116, counters 118, and interfaces 120. The one or more repeat parameters 112 may be associated with one or more of the stimulation settings. For example, a stimulation device may have eight stimulation settings. Each of the eight stimulation settings may have a repeat parameter 112 associated with it. Alternately, a repeat parameter 112 may be associated with a given stimulation setting such as a first stimulation setting. The repeat parameter 112 may cause a given stimulation setting to repeat a number of times in accordance with the repeat parameter 112. Similarly, skip parameters 114 may be associated with one or more of the stimulation settings. Each of the eight stimulation settings may have a skip parameter 114 associated with it. Alternately, a skip parameter 114 may be associated with a given stimulation setting such as a first stimulation setting. The skip parameter 114 may cause a given stimulation setting to be dropped or skipped for a given number of cycles through the array of stimulation settings in accordance with the skip parameter 114. Various other parameters 116 may also be associated with controller 110. In addition, various counters 118 may be associated with controller 110. These counters 118 may be used in determining which pulses or stimulation sets to skip or when to stop repeating a stimulation set. Further, the controller 110 may have various interfaces 120. These interfaces enable communication with the switching circuitry, microprocessor, and pulse generator, among others. These interfaces may take the form of circuitry in the case of a hardware based controller, or they may take the form of software interfaces in the case of a software based controller. Various combinations may be envisaged.

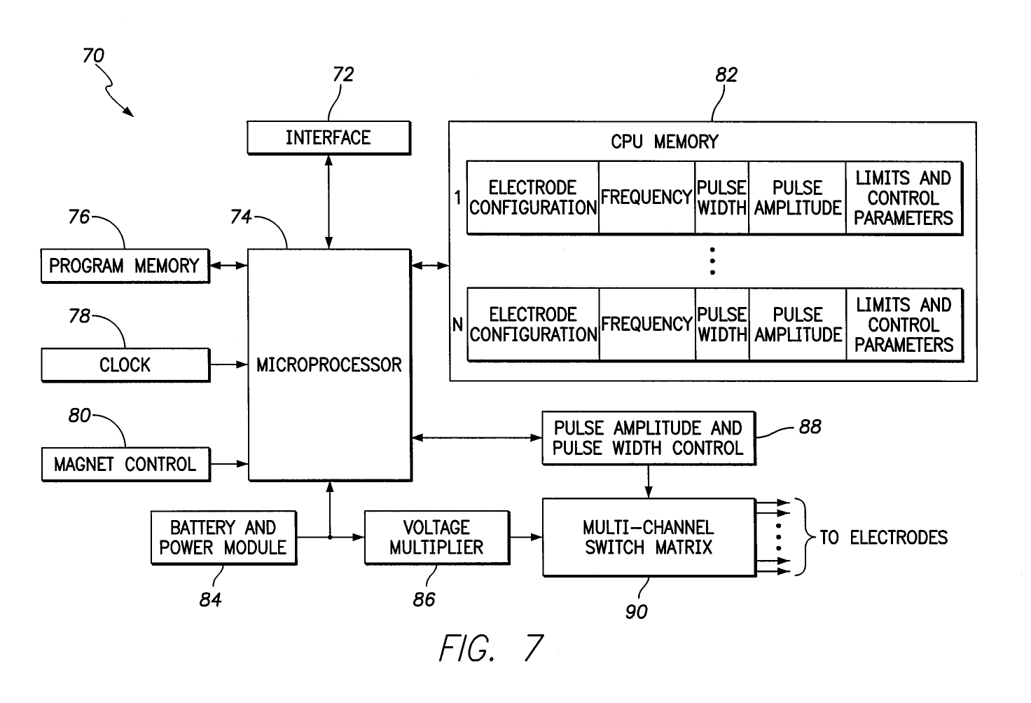

FIG. 7 is a schematic block diagram depicting an exemplary embodiment of a system as seen in FIG. 5. The system 70 has a microprocessor 74, an interface 72, a program memory 76, a clock 78, a magnet control 80, a power module 84, a voltage multiplier 86, pulse amplitude and width control 88, a CPU memory 82, and a multi-channel switch matrix 90. The microprocessor 74 may take the form of various processors and logic circuitry and can function to control pulse stimulations in accordance with settings 1 through N stored in the CPU memory 82. The microprocessor 74 may function in accordance with programs stored in the program memory 76. The program memory 76 may include RAM, ROM, flash memory, and other storage mediums. It may be programmed using the interface 72. The interface 72 may be accessed prior to implanting to program microprocessor 74, program memory 76, and/or CPU memory 82. The interface 72 may include ports or connections to handheld circuitry, computers, keyboards, displays, program storage, or the like. In one preferred embodiment, the interface 72 is configured as a telemetry module that facilitates wireless communication between the system 70 and an external apparatus to deliver programming instructions to the system 70 and to receive measured data and the like from the system 70. The clock 78 may be coupled to the microprocessor 74 and may provide a signal by which microprocessor 74 operates and/or uses in creating stimulation pulses. The magnet control 80 may also interface with the microprocessor 74 and functions to start or stop stimulation pulses. Alternately, a receiver or some other module may be used to accomplish the same task. The system 70 may also have a power supply or battery 84. This power supply 80 may function to power the various circuitries such as the clock 78, microprocessor 74, program memory 76, and CPU memory 82, among others. Further, the power supply 80 may be used in generating the stimulation pulses. As such, the power supply may be coupled to the microprocessor 74, voltage multiplier 86, and/or switch matrix 90. The CPU memory 82 may include RAM, ROM, flash memory, and other storage mediums. The CPU memory 82 may store stimulation settings 1 through N. These stimulation settings may include electrode configuration, pulse frequency, pulse width, pulse amplitude, and other limits and control parameters. The repetition and skipping parameters can be stored in CPU memory 82 and may be associated with each of the stimulation settings 1 through N. The microprocessor 74 may uses these stimulation settings and parameters in configuring the switch matrix 90, manipulating the pulse amplitude and pulse width control 88, and producing stimulation pulses. The switch matrix 90 may permit more than one lead with more than one electrode to be connected to the system 70. The switch matrix 90 may function with other components to selectively stimulate varying sets of electrodes with various pulse characteristics. The controller may be implemented in software for interpretation by microprocessor 74, or a hardware implementation may be coupled to the microprocessor 74, pulse amplitude controller 88, and switch matrix 90.

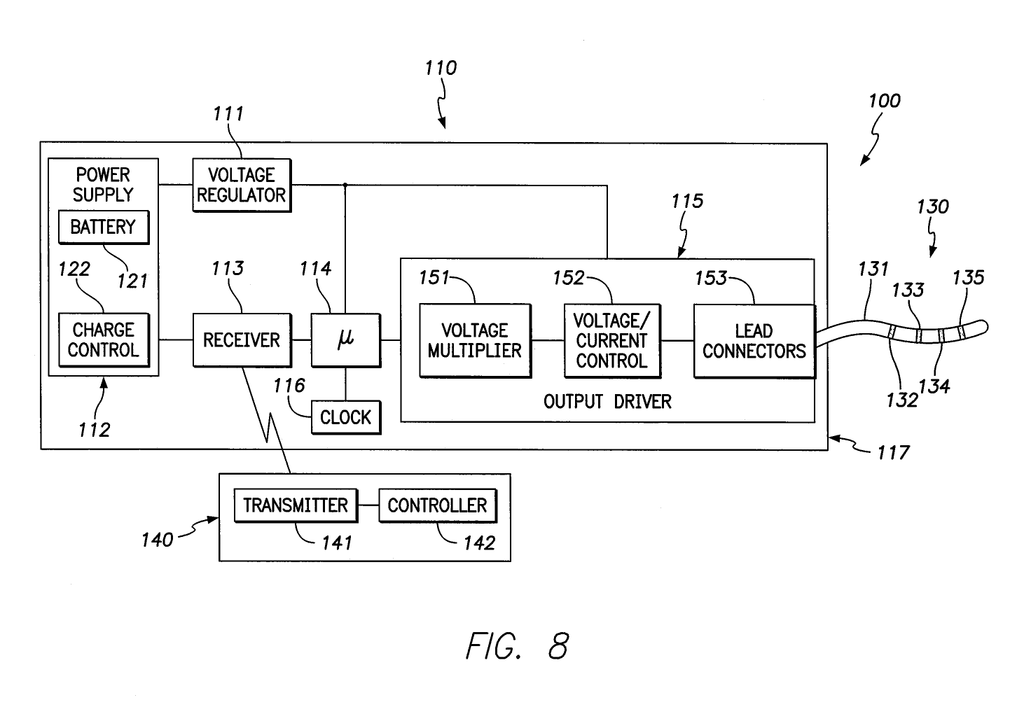

FIG. 8 is a high level block diagram illustrating another example of a neuromodulation system. The system takes the form of an implantable pulse generator (IPG) for generating electrical stimulation for application to a desired area of a body, such as a spinal cord stimulation (SCS) system. The stimulation system 100 of the illustrated embodiment includes a generator portion, shown as an implantable pulse generator (IPG) 110, providing a stimulation or energy source, stimulation portion, shown as a lead 130, for application of the stimulus pulse(s), and an optional external controller, shown as a programmer/controller 140, to program and/or control the implantable pulse generator 110 via a wireless communications link. The IPG 110 may be implanted within a living body (not shown) for providing electrical stimulation from the IPG 110 to a selected area of the body via the lead 130, perhaps under control of the external programmer/controller 140. It should be appreciated that, although the lead 130 is illustrated to provide a stimulation portion of the stimulation system 100 configured to provide stimulation remotely with respect to the generator portion of the stimulation system 100, a lead as described herein is intended to encompass a variety of stimulation portion configurations. For example, the lead 130 may comprise a microstimulator electrode disposed adjacent to a generator portion. Furthermore, a lead configuration may include more (e.g., 8, 16, 32, etc.) or fewer (e.g., 1, 2, etc.) electrodes than those represented in the illustrations. The IPG 110 may comprise a self-contained implantable pulse generator having an implanted power source such as a long-lasting or rechargeable battery (e.g., a primary cell battery). Alternatively, the IPG 110 may comprise an externally powered implantable pulse generator receiving at least some of the required operating power from an external power transmitter, preferably in the form of a wireless signal, which may be radio frequency (RF), inductive, etc.

The IPG 110 of the illustrated embodiment includes a voltage regulator 111, a power supply 112, a receiver 113, a microcontroller (or microprocessor) 114, an output driver circuitry 115, and a clock 116. The power supply 112 provides a source of power, such as from a battery 121 (the battery 121 may comprise a non-rechargeable (e.g., single use) battery, a rechargeable battery, a capacitor, and/or like power sources), to other components of the IPG 110, as may be regulated by the voltage regulator 111. The charge control 122 provides management with respect to the battery 121. The receiver 113 provides data communication between the microcontroller 114 and the controller 142 of the external programmer/controller 140, via the transmitter 141. It should be appreciated that although the receiver 113 is shown as a receiver, a transmitter and/or transceiver may be provided in addition to or in the alternative to the receiver 113, depending on the communication links desired. The receiver 113, in addition to or in the alternative to providing data communication, provides a conduit for delivering energy to the power supply 112, such as where RF or inductive recharging of the battery 121 is implemented. The microcontroller 114 provides control with respect to the operation of the IPG 110, such as in accordance with a program provided thereto by the external programmer/controller 140. The output driver circuitry 115 generates and delivers pulses to selected ones of electrodes 132-135 under control of the microcontroller 114. For example, the voltage multiplier 151 and voltage/current control 152 may be controlled to deliver a constant current pulse of a desired magnitude, duration, and frequency to a load present with respect to particular ones of the electrodes 132-135. The clock 116 preferably provides system timing information, such as may be used by the microcontroller 114 in controlling system operation, as may be used by the voltage multiplier 151 in generating a desired voltage, etc.

The lead 130 of the illustrated embodiment includes a lead body 131, preferably incarcerating a plurality of internal conductors coupled to lead connectors (not shown) to interface with the lead connectors 153 of the IPG 110. The lead 130 further includes electrodes 132-135, which are preferably coupled to the aforementioned internal conductors. The internal conductors provide electrical connection from individual lead connectors to each of a corresponding one of the electrodes 132-235. In the exemplary embodiment, the lead 130 is generally configured to transmit one or more electrical signals from the IPG 110 for application at, or proximate to, a spinal nerve or peripheral nerve, brain matter, muscle, or other tissue via the electrodes 132-135. The IPG 110 is capable of controlling the electrical signals by varying signal parameters such as intensity, duration and/or frequency in order to deliver a desired therapy or otherwise provide operation as described herein. The lead (stimulation portion) and IPG (generator portion) may comprise a unitary construction, such as that of a microstimulator configuration.

As mentioned above, the programmer/controller 114 provides data communication with the IPG 110, such as to provide control (e.g., adjust stimulation settings), provide programming (e.g., alter the electrodes to which stimulation pulses are delivered), etc. Accordingly, the programmer/controller 114 of the illustrated embodiment includes a transmitter 141, for establishing a wireless link with the IPG 110, and a controller 142, to provide control with respect to the programmer/controller 114 and IPG 110. Additionally or alternatively, the programmer/controller 114 may provide power to the IPG 110, such as via the RF transmission by transmitter 141. Optionally, however, a separate power controller may be provided for charging the power source within the IPG 110.

The two examples of neuromodulation systems as shown in FIGS. 3-7 and 8 include various features that may be selectively implemented in a system to be used to provide nerve stimulation for treating hypertension and other cardio-renal disorders of a patient. Additional details with respect to pulse generation systems and the delivery of stimulation pulses and patterns may be found in U.S. Pat. Nos. 6,609,031, 7,228,179, and 7,571,007, the entire disclosures of which are incorporated herein by reference. The next section describes additional features to be implemented in the system to control the neural modulation based on results obtained by monitoring one or more parameters of the patient related to the treatment, which may be physiological parameters such as blood pressure, blood volume, and the like.

Control of Neural Modulation by Monitoring Patient Condition

Aside from manually controlling the therapy, a feedback regulation can be used as a trigger to start, to stop, or to modify the neuromodulation. Such a feedback loop could be dependent on one or more of: optical volumetric monitoring using, e.g., a PPG (photoplethysmograph) sensor, electrical volumetric monitoring using, e.g., impedance, and pressure measurements using, e.g., a LAP (left atrial pressure) sensor or an integrated RADI pressure sensor.

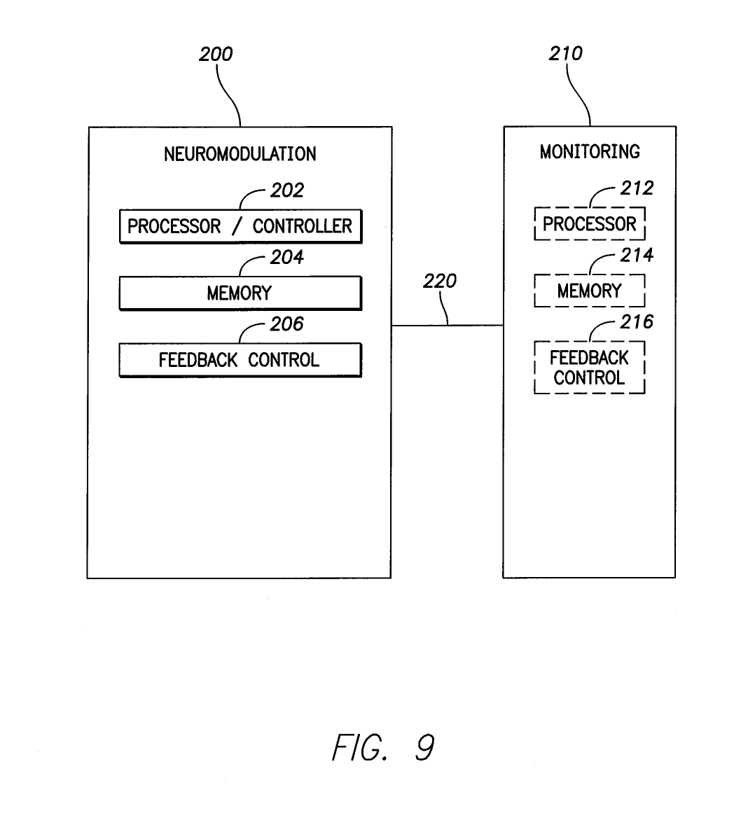

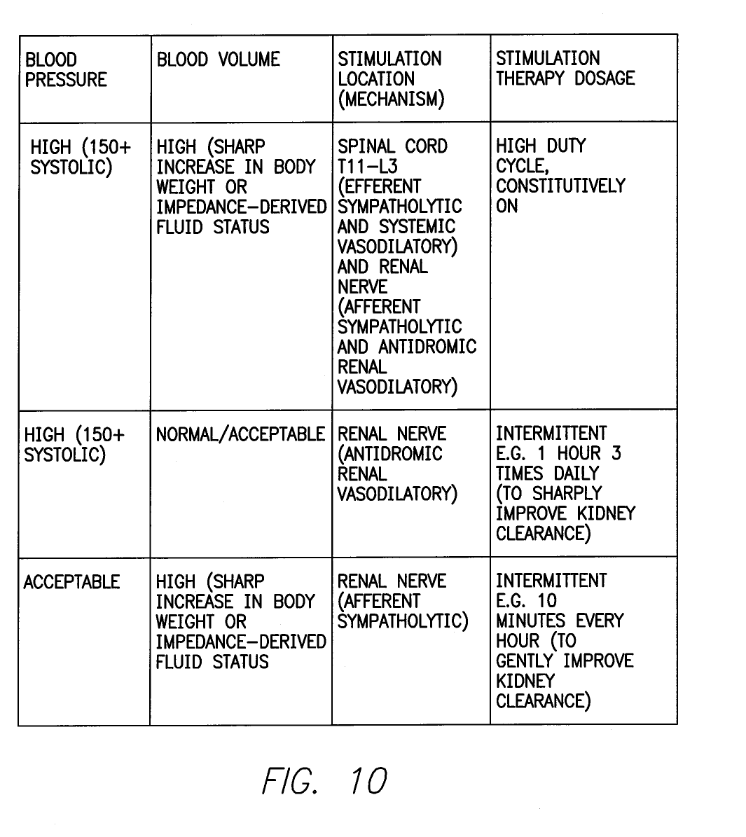

FIG. 9 is a schematic diagram illustrating an example of a feedback system for controlling the neural modulation to treat hypertension based on results of a monitoring device. A neuromodulation system 200 receives monitoring device data from the monitoring device 210 via a communication link 220 which may be wired or wireless. The neuromodulation system 200 may be implemented as the system 70 of FIG. 7 or the system 110 of FIG. 8, or it may include a combination of features selected from those systems. FIG. 9 shows the system 200 includes a neural modulation processor or controller 202, a memory 204, and a feedback control module 206. The processor 202 controls the delivery of neuromodulation signals such as pulses to the electrodes (similar to the microprocessor 74 of FIG. 7 and the microcontroller 114 of FIG. 8). The other components of the system 200 (which can be selected from FIGS. 7 and 8, for example) are omitted for simplicity.

In one exemplary system, the monitoring device 210 is a pressure sensor and/or a volume sensor implemented by way of an implantable medical device, such as CRT-D (Cardiac Resynchronization Therapy Defibrillator). The data from the monitoring device 210 is processed by the feedback control module 206. Based on the data, the feedback control module 206 provides feedback control data to the processor 202 to be used for controlling/adjusting the neural modulation. For example, a direct measure such as left atrial pressure, or an indirect measure such as left atrial volume or lung fluid volume estimates using cardiac and thoracic impedance, may serve as the control signal. When the pressure or volume (or their indirect surrogates) exceeds a certain threshold, a signal is given to the neural modulation processor 202 to turn on and begin the state to drive blood pressure and systemic volumes down. When the pressure or volume returns to acceptable limits, the signal is removed from the neural modulation processor 202 and the control state is turned off.

In FIG. 9, the feedback control module 206 is part of the neuromodulation system 200. In other embodiments, the feedback control module 206 may be separate from the neuromodulation system 200. For example, as shown in broken lines in FIG. 9, the monitoring device 210 may include a data processor 212 and a memory 214 to store and process monitored data, and to execute a feedback control module 216 using the monitored data.

In another embodiment, the monitoring device 210 is provided as external monitoring equipment which communicates with the neuromodulation system 200 preferably via a wireless link 220. A telemetry unit will be provided as mentioned above. The pressure and/or volume monitoring is performed using the external monitoring equipment 210. The data is sent to the feedback control module 206 via the link 220. Alternatively, the feedback control module 216 is provided externally (e.g., as part of the external monitoring equipment 210), the desired state of the neuromodulation is determined by external software, and the control signal (e.g., ON/OFF) is to be generated and transmitted by wireless communication to the implanted nerve modulator including the neuromodulation system 200. In this case, the “external monitoring equipment” can be a blood pressure cuff and a body weight scale. The desired state of ON occurs when the arm blood pressure increases (e.g., 150/90) and/or when the body weight increases (e.g., 6 pounds in 3 days), while an OFF state occurs when the arm blood pressure and/or body weight and/or change in body weight are within acceptable limits for the patient. The neural modulator may be, for example, a spinal cord modulator or a renal nerve modulator. In addition, the neural modulator may be in telemetric communication with an external transmitter such as a Merlin@home unit that transmits information obtained from the neural modulator to the patient's medical care provider.

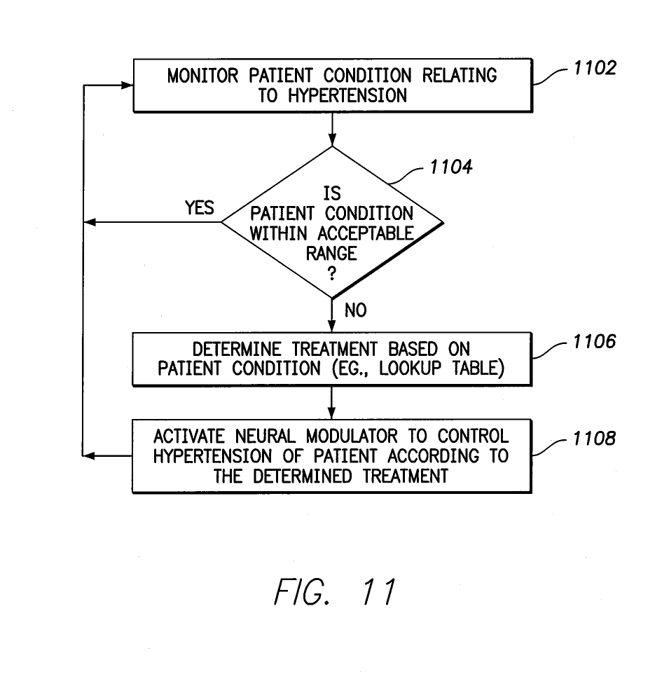

In an ideal system, delivery of neuromodulation to achieve blood pressure control via one or more of the various mechanisms described herein can be controlled with great precision. FIG. 10 shows an example of a neuromodulation delivery table that summarizes the various scenarios expected to be encountered and the system response for delivery of neuromodulation. The scenarios are characterized by the conditions of the patient and neuromodulation options. In the example shown, the conditions are presented in terms of monitored blood pressure (e.g., high or acceptable) and blood volume (e.g., high or normal/acceptable). Based on the monitored blood pressure and monitored blood volume, the modulation location of each neural modulator is specified. The modulation location typically determines the mechanism by which the renal sympathetic hyperactivity of the patient is decreased and/or the renal blood flow is increased and/or the peripheral systemic vascular resistance is decreased. In response to each scenario, a modulation therapy dosage is determined. The therapy dosage specifies one or more electric activation parameters including, for example, a current level, a pulse width, a frequency, and a duty cycle. As the condition of the patient changes, the system response can be adjusted based on the feedback. The control signal will not simply be ON/OFF, but will contain information specifying the one or more electric activation parameters described above. The neuromodulation delivery table may be stored in the memory 204 or 214 to be used by the feedback control module 206 or 216 for determining the neuromodulation delivery based on the monitoring device data.

FIG. 11 is a flow diagram illustrating an example of feedback control of neuromodulation for hypertension control. In step 1102, the monitoring device 210 monitors the patient condition relating to hypertension (e.g., blood pressure, blood volume, etc.). In step 1104, the feedback control module 206 or 216 determines whether the patient condition is within an acceptable range. If yes, the process returns to step 1102. If no, the feedback control module 206 or 216 determines the appropriate treatment based on the patient condition (step 1106). One example employs a look up table such as the neuromodulation delivery table of FIG. 10. In step 1108, the processor/controller 202 activates the neural modulator to control hypertension of the patient according to the determined treatment (step 1108).

In the description, numerous details are set forth for purposes of explanation in order to provide a thorough understanding of the present invention. However, it will be apparent to one skilled in the art that not all of these specific details are required in order to practice the present invention. It is also noted that the invention may be described as a process, which is usually depicted as a flowchart, a flow diagram, a structure diagram, or a block diagram. Although a flowchart may describe the operations as a sequential process, many of the operations can be performed in parallel or concurrently. In addition, the order of the operations may be re-arranged.

From the foregoing, it will be apparent that the invention provides methods, apparatuses and programs stored on computer readable media for delivering neural modulation to control hypertension and other cardio-renal disorders. Additionally, while specific embodiments have been illustrated and described in this specification, those of ordinary skill in the art appreciate that any arrangement that is calculated to achieve the same purpose may be substituted for the specific embodiments disclosed. This disclosure is intended to cover any and all adaptations or variations of the present invention, and it is to be understood that the terms used in the following claims should not be construed to limit the invention to the specific embodiments disclosed in the specification. Rather, the scope of the invention is to be determined entirely by the following claims, which are to be construed in accordance with the established doctrines of claim interpretation, along with the full range of equivalents to which such claims are entitled.

What is claimed is:

1. A method of treating hypertension, the method comprising:
monitoring a blood pressure of a patient;
delivering a neuromodulation device to a patient's body; and
applying an electric activation using the neuromodulation device to decrease renal sympathetic hyperactivity of the patient based on the monitored blood pressure of the patient, substantially without thermal energization of the patient's body by applying the electric activation.

2. The method of claim 1, further comprising:
selecting at least one target of the following three targets to which the electric activation is applied to activate: (i) a region of a spinal cord responsible for renal innervation, or (ii) neurons in a vicinity of a kidney, or (iii) a region of the spinal cord or a peripheral nerve to produce vasodilation with reduced systemic vascular resistance; and
based on the selected at least one target, determining at least one of a plurality of electric activation parameters to be used in applying the electric activation, including a current level, a pulse width, a frequency, a duty cycle, and a location of the patient's body to which the electric activation is applied.

3. The method of claim 1, further comprising:
selecting at least one of the following three types of electric activation to be applied: (i) sympathetic inhibition of renal innervation, or (ii) antidromic activation at a kidney level of the patient to increase renal blood flow, or (iii) antidromic activation at a periphery of the patient to reduce systemic vascular resistance; and
based on the selected type of electric activation, determining at least one of a plurality of electric activation parameters to be used in applying the electric activation, including a current level, a pulse width, a frequency, a duty cycle, and a location of the patient's body to which the electric activation is applied.

4. The method of claim 1, further comprising:
adjusting the electric activation based on the monitored blood pressure of the patient.

5. The method of claim 4,
wherein adjusting the electric activation comprises modifying at least one of a plurality of electric activation parameters including a current level, a pulse width, a frequency, a duty cycle, and a location of the patient's body to which the electric activation is applied.

6. The method of claim 1, further comprising:
monitoring a blood volume of the patient; and
adjusting the electric activation based on the monitored blood pressure and the monitored blood volume of the patient.

7. The method of claim 6,
wherein adjusting the electric activation comprises modifying at least one of a plurality of electric activation parameters including a current level, a pulse width, a frequency, a duty cycle, and a location of the patient's body to which the electric activation is applied.

8. The method of claim 6,
wherein adjusting the electric activation comprises referring to a lookup table which provides electric activation plans for different conditions of monitored blood pressure and monitored blood volume, the electric activation plans each specifying a setting of at least one of a plurality of electric activation parameters including a current level, a pulse width, a frequency, a duty cycle, and a location of the patient's body to which the electric activation is applied.

9. The method of claim 6,
wherein the electric activation is applied until the monitored blood pressure falls within a preset blood pressure range and the monitored blood volume falls within a preset blood volume range for the patient.

10. A system of treating hypertension, the system comprising:
a neuromodulation device configured to be delivered to a patient's body and to apply an electric activation to decrease renal sympathetic hyperactivity of the patient based on monitored blood pressure of the patient, substantially without thermal energization of the patient's body by applying the electric activation.

11. The system of claim 10, further comprising:
a processor;
a memory; and
a feedback control module to provide feedback control information for adjusting the electric activation to be applied by the neuromodulation device based on the monitored blood pressure of the patient.

12. The system of claim 11,
wherein adjusting the electric activation comprises modifying at least one of a plurality of electric activation parameters including a current level, a pulse width, a frequency, a duty cycle, and a location of the patient's body to which the electric activation is applied.

13. The system of claim 12,
wherein the electric activation is adjusted based on which of the following three targets to which the electric activation is applied to activate: (i) a region of a spinal cord responsible for renal innervation, or (ii) neurons in a vicinity of a kidney, or (iii) a region of the spinal cord or a peripheral nerve to produce vasodilation with reduced systemic vascular resistance.

14. The system of claim 12,
wherein the electric activation is adjusted based on which of the following three types of electric activation is to be applied: (i) sympathetic inhibition of renal innervation, or (ii) antidromic activation at a kidney level of the patient to increase renal blood flow, or (iii) antidromic activation at a periphery of the patient to reduce systemic vascular resistance.

15. The system of claim 11,
wherein the neuromodulation device is configured to apply the electric activation to decrease renal sympathetic hyperactivity of the patient based on monitored blood pressure and monitored blood volume of the patient, substantially without thermal energization of the patient's body by applying the electric activation; and
wherein the feedback control module provides feedback control information for adjusting the electric activation to be applied by the neuromodulation device based on the monitored blood pressure and monitored blood volume of the patient.

16. The system of claim 15,
wherein adjusting the electric activation comprises modifying at least one of a plurality of electric activation parameters including a current level, a pulse width, a frequency, a duty cycle, and a location of the patient's body to which the electric activation is applied.

17. The system of claim 16,
wherein adjusting the electric activation comprises referring to a lookup table which provides electric activation plans for different conditions of monitored blood pressure and monitored blood volume, the electric activation plans each specifying a setting of at least one of a plurality of electric activation parameters including a current level, a pulse width, a frequency, a duty cycle, and a location of the patient's body to which the electric activation is applied.

18. The system of claim 15,
wherein the neuromodulation device is configured to apply the electric activation until the monitored blood pressure falls within a preset blood pressure range and the monitored blood volume falls within a preset blood volume range for the patient.

19. A computer-readable storage medium storing a plurality of instructions for controlling a data processor to provide treatment for hypertension of a patient via a neuromodulation device, the plurality of instructions comprising:
instructions that cause the data processor to apply an electric activation using the neuromodulation device to decrease renal sympathetic hyperactivity of the patient based on monitored blood pressure of the patient, substantially without thermal energization of the patient's body by applying the electric activation.

20. The computer-readable storage medium of claim 19, wherein the plurality of instructions further comprise:
instructions that cause the data processor to provide feedback control information for adjusting the electric activation to be applied using the neuromodulation device based on the monitored blood pressure of the patient.
